(19) United States
(12) Patent Application Publication
(10) Pub. No.: US 2007/0173824 A1
(43) Pub. Date: Jul. 26, 2007

(54) Method of percutaneous paracoccygeal pre-sacral stabilization of a failed artificial disc replacement

(76) Inventor: Charles D. Rosen, Manhattan Beach, CA (US)

Correspondence Address: FULWIDER PATTON LLP, Suite 1550, 200 Oceangate, Long Beach, CA, 90802, US

(21) Appl. No.: 11/335,267

(22) Filed: Jan. 19, 2006

(51) Int. Cl. A61F 2/30

(52) U.S. Cl. 606/61

(57) ABSTRACT

A procedure for stabilization in situ of a failed artificial disc replacement (ADR) using a pre-sacral paracoccygeal approach to an inter-vertebral disc space, such as the L5-S1 disc space for example, where a bore is created in the ADR using a drill, and then a fastener is inserted into the bore of the ADR and used to compress the endplates of the ADR. The fastener may have ends that prevent movement of the fastener once established in the ADR, and maintain the ADR in compression. Subsequent to the stabilization of the ADR, a spinal fusion operation can be performed with the stabilized ADR such that regenerative growth of bone can surround and form over the ADR without relative movement of the ADR to resist complete fusion and immobilization, and thus to improve the clinical results.

### BACKGROUND OF THE INVENTION

The present invention relates generally to spinal column reconstruction procedures, and more particularly to a procedure for stabilizing an artificial disc replacement (ADR) in situ using a percutaneous paracoccygeal pre-sacral approach. This is performed for the specific purpose of improving the clinical results of a concurrently performed posterior fusion in the situation where an ADR has failed.

Lumbar disc replacement surgery has recently become an available alternative to lumbar spine fusion, although the development of the procedures and the prostheses themselves for use in the United States are in their infancy. Presently, disc replacement surgery is proposed only for single-level, painful degenerative disc disease that has failed to improve after at least six months of intense spine-focused rehabilitation in a patient without significant physical or psychological contraindications. Candidates are presently diagnosed with degenerative disc disease (DDD) or post-laminectomy syndrome at either the L4-L5 or L5-S1 levels of the lumbar spine, but not both, although other levels of the spine are also theoretically possible.

Artificial discs, such as the Charite™ artificial disc manufactured by DePuy Spine, Inc., 325 Paramount Drive Raynham, Mass. 02767, were approved by the FDA in October, 2004. The object of the artificial disc is to restore the intervertebral disc height and neuroforaminal height while restoring physiologic motion. The disc insertion is performed anteriorly through a small incision in the abdomen. The patient's organs are displaced to the side so that the surgeon can visualize the spine while shielding important anatomic structures. The collapsed or degenerated disc is removed and the prosthetic artificial disc is inserted in the spinal column in its place. The prosthesis is formed of two metal plates made of a cobalt chrome alloy or other suitable biocompatible material sandwiching a plastic (ultra-high molecular weight polyethylene or UHMWPE) core. During the replacement procedure, the two endplates are pressed into the vertebrae above and below the disc space. The end plates are formed with teeth on the outer surface that help secure the prosthesis to the adjoining bone. The plastic core and endplates serves to restore the proper distance between the two vertebrae (disc height), and simulate the resiliency of the natural disc. The theory behind the disc replacement surgery is that the artificial disc stays in place by the spinal ligaments and remaining part of the annulus of the disc, as well as the compressive force of the spine.

Unfortunately, the success rate of the ADR surgery has been less than optimal, with a large percentage of ADR patients experiencing severe and chronic pain after the surgery. The present inventor voiced doubts at the time the FDA approved the ADR about the safety and reliability of the new disc replacement surgery, doubts that have become realized by the large number of patients who have experienced tremendous pain and complications with their new disc replacements. One major complication experienced by a large majority of patients is that the disc fails to bond properly in the spinal column, resulting in instability or dislocation/subluxation of the disc and the accompanying disabilitating pain. The ADR may increase the motion of the facet joints, leading to subsequent degeneration and pain. Fractures of various parts of the vertebra may also occur during or after the implantation, as well as fractures of the polyethylene core. Some cases of chronic debilitating pain may not have any obvious cause but still constitute a failure of the ADR. The widespread failure of these discs has become so prevalent that it became apparent to the present inventor that a better salvage procedure was needed where the disc is stabilized in some fashion prior to a posterior fusion being attempted. Removal of the ADR is a poor and dangerous alternative due to the life threatening consequence of exsanguination and death from tearing of scarred down large vessels. Thus, stabilization by the method of the present invention was developed to increase the clinical success rate of a salvaging fusion procedure done posteriorly.

The purpose of the stabilization procedure is to allow for a posterior fusion. A posterior fusion is attempted by using bone graft or bone substitutes to promote the vertebra to fuse together. Presently, when a fusion has been attempted for a failed ADR the results have been poor with a sixty percent (60%) failure rate defined as continuing pain. Sometimes fusion occurs and pain is still present, and many other times fusion is unsuccessful. Without the ADR, posterior fusion has a success rate of over eighty percent (80%), so the presence of the ADR has a dramatic effect on the success rate of the fusion surgery. The present inventor has proposed a safe procedure to dramatically increase the success rate of the posterior fusion when an ADR is present.

### SUMMARY OF THE INVENTION

The present invention proposes that a stabilization of the ADR prior to attempting a posterior fusion will promote the fusion process by encouraging regenerating bone material to grow around the ADR and fortify the spine structure. Stabilization of a floating or loose ADR is performed percutaneously by a small diameter drill. The approach to the lumbar spine is paracoccygeal in the area posterior to the mesorectum and anterior to the sacrum to avoid the scarred area of the iliac vessels. The L5-S1 disc space, for example, can be accessed by drilling through a cannula that protect the rectum and intestines. This same method described here can be used for the L4-L5 or L3-L4 space as well. The drill is used to pierce the metallic base plates of the ADR to create a through and through bore, with irrigation maintaining a proper environment at the drilling surface. Suction and evacuation of the debris generated by the drilling operation could also be conducted simultaneously with the drilling. After drilling through the ADR, a fastener is placed into the bore of the disc to compress the disc in situ and stabilize the disc in the spinal column. Subsequently, a spinal fusion is performed to allow regenerative bone to envelope the stabilized disc and thus permanently address the instability or other causes noted above that may be the root of pain from the failed ADR.

### BRIEF DESCRIPTION OF THE DRAWINGS

Other features and advantages of the invention will become apparent from the following detailed description, taken in conjunction with the accompanying drawings which illustrate, by way of example, the features of the invention:

FIG. 1 is a lateral view, partially in shadow, of a patient prone on the table with fluoroscopes in place and guidewire needle inserted;

FIG. 2 is a view of the insertion of the blunt trocar into the S1 disc space;

FIG. 3 is a top view of the insertion of the blunt trocar into the S1 disc space;

FIG. 4 is a side view of the insertion of the drill into the ADR;

FIG. 5 is a top view of the insertion of the drill into the ADR;

FIG. 6 is a perspective view of the L5-S1 disc space with the ADR in place;

FIG. 7 is a perspective view of the L5-S1 disc space with the ADR being drilled;

FIG. 8 is a perspective view of the L5-S1 disc space after the drilling operation;

FIG. 9 is a perspective view of the L5-S1 disc space with a fastener being inserted into the bore of the ADR;

FIG. 10 is a perspective view of the L5-S1 disc space with a first embodiment of a fastener inserted in the bore and the proximal end being screwed into position;

FIG. 11 is a perspective view of the L5-S1 disc space with a first embodiment of a fastener inserted into the bore and the proximal end tightened to place the ADR in compression;

FIG. 12 is an enlarged perspective view of the first embodiment of the fastener in the undeployed and deployed positions;

FIG. 13 is an enlarged perspective view of a second embodiment of the fastener in the undeployed and deployed positions; and

FIG. 14 is a perspective view of the L5-S1 disc space with a third embodiment of a fastener inserted into the bore of the ADR.

### DETAILED DESCRIPTION OF THE PREFERRED EMBODIMENTS

Described below is a procedure for in situ stabilization of a failed ADR prior to a posterior fusion procedure. The stabilization employs a novel paracoccygeal percutaneous approach that is far safer than an anterior approach and permits greater fusion opportunity due to immobilization of the failed ADR. Prior to stabilization, it may be preferable to employ a postero-lateral approach described herein where it has been determined that there is a need to retrieve a dislocated or subluxed ADR prior to stabilization, or because direct visualization is desired through the endoscope of the concurrent stabilization procedure through the pre-sacral approach. Access through one or more poster-lateral portals may also assist also in evacuating debris that results from the drilling procedure and in cooling the drill with irrigation.

Percutaneous posterolateral endoscopic access to a failed ADR disc space requires initially the establishment of key fluoroscopic landmarks using the fluoroscopes in the AP and lateral plane. These landmarks are the center of the disc, the area of the disc centered just lateral to the pedicle, and the disc angle line that bisects the disc in the lateral projection. The skin entry is determined from the inclination of the failed disc. The lateral location of the skin incision's from the midline determines the trajectory angle into the particular disc space of the ADR just lateral to the pedicle, the basal part of each side of the neural arch of a vertebra connecting the laminae with the body.

A long guide wire is laid across the patient in the anteroposterior (AP) plane and the fluoroscope is used to locate the midline of the disc in the AP plane. A pen mark is placed on the skin is used to demarcate the position. Then the guidewire is placed transversely over the disc and this is position is also marked. The intersection of the lines is the center of the disc. It is important to obtain a true AP line and a true lateral line of the selected disc space being visualized so that both endplates of the ADR are precisely parallel. The entry point for a spinal needle is then estimated to be on the axis of the transverse line with a trajectory of about 30 to 40 degrees off the midline. At L5/S1 juncture, the angle of attack may be steeper to avoid the iliac crest. This estimation is roughly 4 fingerbreadths lateral of the midline. However, the main way of guiding the needle to enter the disc just lateral to the facet joint is by tracking the progress of the needle using the fluoroscopes and re-adjusting the trajectory as needed in both planes until the desired location is hit. In some instances, hitting the facet and “walking off” the needle laterally can be helpful and confirmatory of location. Monitoring with intra-operative continuous evoked potentials and EMG's help to prevent inadvertently injuring the nerve root, which can be employed with EMG feedback prior to entering the disc (such as done with pedicle screw testing).

The hollow needle with a stylet is advanced into the disc space of the ADR. Once the needle is in place and the location confirmed, a guide wire is then exchanged for the stylet. A blunt dilator is then advanced over the guidewire, and a working cannula is advanced over the guidewire. The blunt dilator and guide wire are removed once the working cannula is sufficiently deep into the annulus. The endoscope is then inserted into the working cannula for visualization. Subsequent work to reduce the disc, if needed, can be done either through the same cannula as the endoscope, or through a separate and identical portal to the disc but on the other side. A cannula portal on the other side established in the same fashion can be used to remove debris from the drilling operation or to provide irrigation for same if necessary.

To reduce a dislocated or subluxed ADR, a number of methods may be tried with the simplest first. A sharp claw may be set into the polyethylene midsection of the disc or latched on the metal plate in effort to pull the ADR back into place. If this fails, then an acrylic glue may be applied to the ADR since such a glue can be adherent to polyethylene. A small amount of glue is pushed through a spinal needle, or the like, that is in contact with the ADR and allowed to set, then reduction attempted by manipulating the spinal needle with the adhered ADR. Failing this, the ADR may be penetrated by drilling and screwing a threaded member into the ADR. For example, a threaded sharp trocar point guide wire may be used to attempt to insert directly into the polyethylene. If penetration is difficult then drilling first may be needed. If the metal endplate is also dislocated or subluxed, then initial drilling will almost certainly be required.

Whether reduction of a dislocated ADR is required initially or not, the postero-lateral portal(s) can be used to assist in the stabilization of the ADR that will commence through the pre-sacral approach and portal concurrently. The accessory postero-lateral portal(s) is useful for visualization of the progress of the stabilization. Suction for debris removal as well as irrigation can be accomplished using one portal for each. If only one accessory portal is used, then intermittent or continuous suction and irrigation can be done simultaneously through therein.

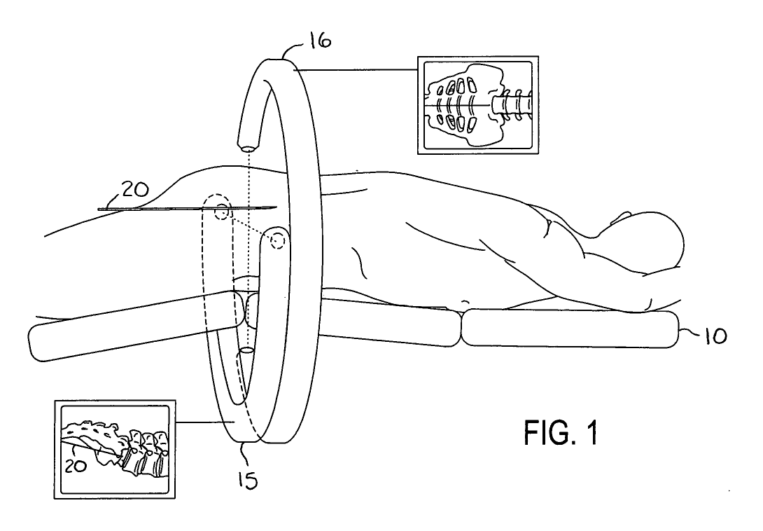

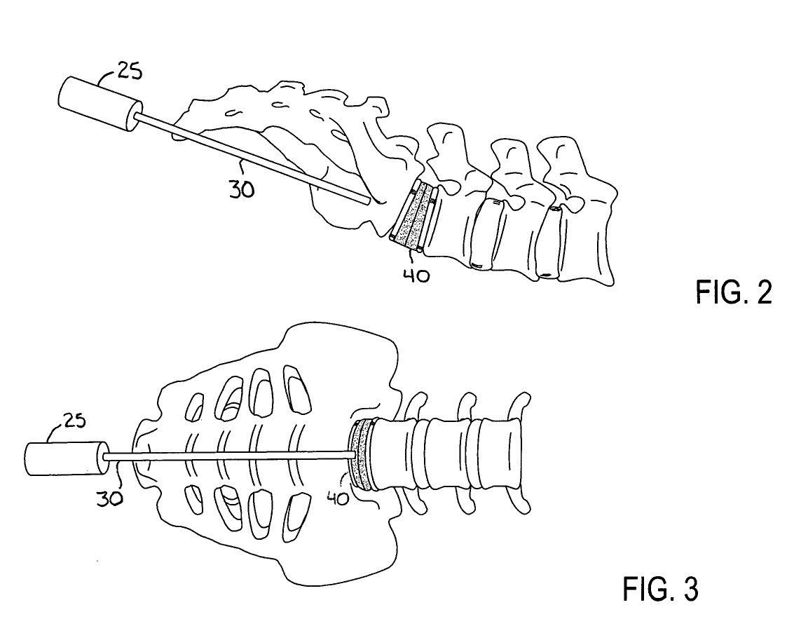

The procedure for stabilizing the ADR in situ will now be described. First, the pre-sacral approach to the ADR stabilization is initiated by prepping the area around the patient's anus with a betadine wash and then antiseptic paint. The area is draped off, and a standard surgical prep of the sacrococcygeal area and lumbar spine area are performed. As shown in FIG. 1, the patient is prone on a Jackson table or similar table 10 with a slight flexion of hips to improve the exposure of the sacrococcygeal area. First and second C-arm fluoroscopes 15,16 are positioned such that the first fluoroscope 16 is aligned in AP plane and the second fluoroscope 15 is aligned in lateral plane. Once the scopes are in place and their orientations confirmed, a 1.5 to 2.0 cm incision is made through the skin and subcutaneous fascia 1-2 cm caudal to the left or right of the tip of the coccyx and 2 cm superior to it. A cannula and blunt trocar is passed through the incision and located using the fluoroscopes to the L5-S1 disc area. As shown in FIGS. 2 and 3, a blunt trocar 25 and cannula 30 is inserted through the incision until the distal end of the cannula is positioned on the anterior midline of sacrum. At this point, the fluoroscopes in the AP and lateral planes are checked and the position of the blunt trocar and cannula are confirmed.

Once the fluoroscopes are checked, the blunt trocar and working cannula are advanced along the anterior sacrum with care to maintaining constant contact with the skeletal structure up to a position just below L5/S1 disc space. The trajectory of the blunt trocar and the cannula are once again confirmed using the AP and Lateral fluoroscopy. At this point, the blunt trocar is retracted and replaced with sharp guide pin that is used to tap into the sacrum until it reaches the base plate of failed ADR 40.

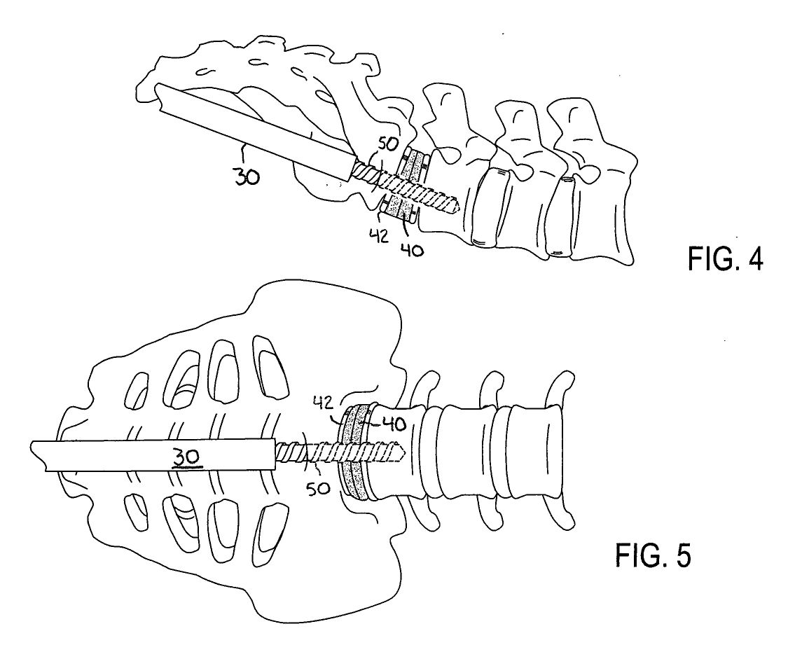

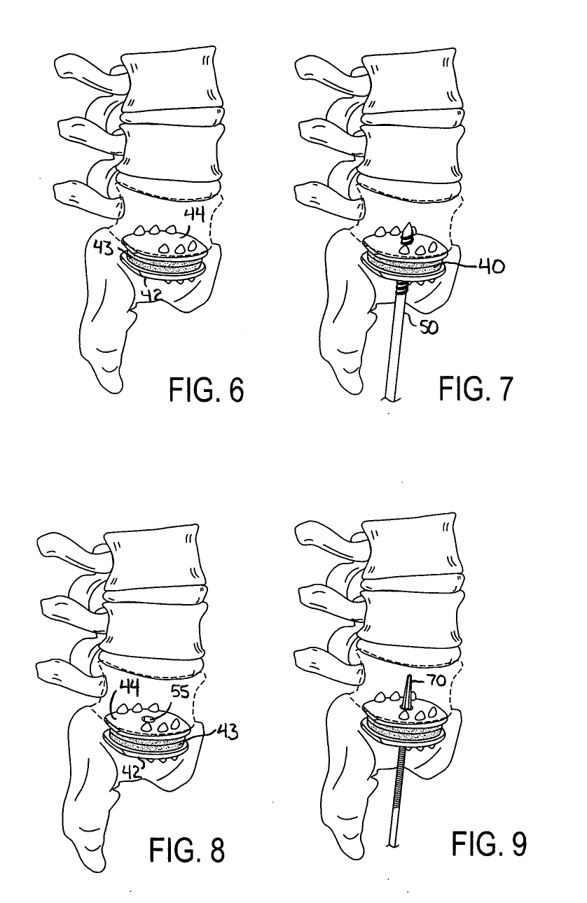

It is preferable at this point to dilate the soft tissue and boney entry at the sacrum with dilators in 2 mm increments, beginning with 6 mm and concluding with a 10-12 mm working cannula that is docked into the sacrum. With the entrance to the sacrum dilated, a drill 50 (see FIGS. 4 and 5) is inserted into the cannula 30 until it bears against the endplate 42 of the ADR 40. Checking and confirming the orientation of the drill 50 so as to be orthogonal, or within 45 degrees of this, to the plane of the ADR end plate 42 and centered in the face of the endplate, or off center as long as projected trajectory includes both metal plates of the ADR, the drill 50 is actuated to penetrate the ADR 40, creating a bore 55 through both endplates 42, 44 as well as the interior portion 43 of the ADR as shown in FIGS. 6 and 7. During the drilling operation, irrigation can be applied through a postero lateral portal and suction to remove debris from the drilling can be removed concurrently or alternatively through the same or a second portal.

After the bore 55 in the ADR 40 has been established (see FIG. 8), the ADR is compressed to arrest the relative movement of the endplates 42, 44 and stabilize the disc. The stabilization and compression can be achieved by withdrawing the drill and advancing a fastener 70 though the cannula and into the just created bore in the ADR (FIG. 9). The fastener 70 may have fixed directional fins (not shown) that permit movement in a first direction but resist movement in a second direction, or the fastener may have spring loaded prongs that expand once the fastener passes through the distal end plate 44 of the ADR 40 to prevent the fastener from retracting back through the bore 55.

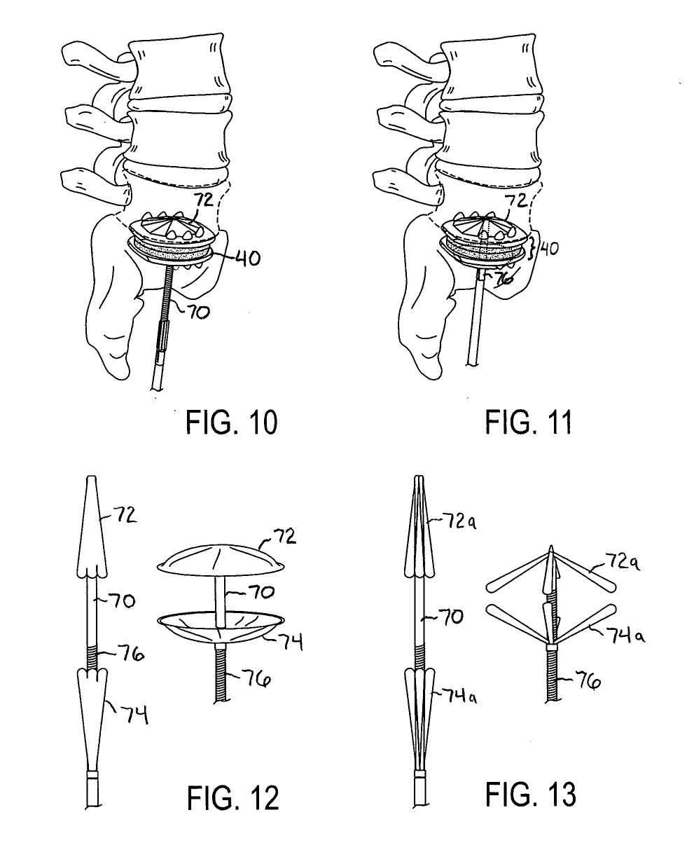

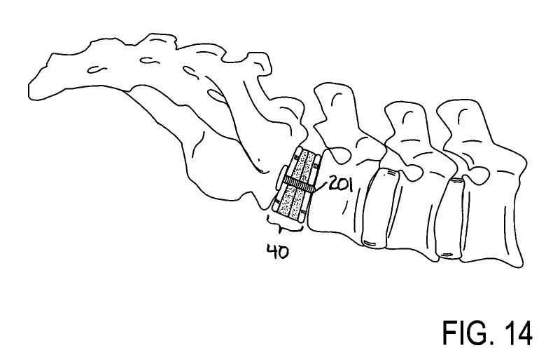

FIGS. 10 and 11 illustrate one example of a fastener 70 (shown in FIG. 12) that could be employed with the present invention, where a fastener 70 including an umbrella distal end 72 and umbrella proximal end 74 is passed through the bore 55 in the ADR and then the umbrella portion 72 is deployed to fix the fastener 70 in the ADR 40. The fastener 70 includes a threaded portion 76 that cooperates with a threaded portion on the proximal end to compress the ADR between the distal end and the proximal end. FIG. 13 illustrates an alternative embodiment of the fastener 70a using the umbrella concept with distal end 72a and proximal end 74a. Another alternate embodiment uses magnets in deployable prongs on the fastener that are drawn out of the fastener by the metallic end plate once passed through the bore 55 so as to bear against the end plate when the fastener is tightened. FIG. 14 illustrates yet another embodiment with a threaded fastener used after drilling with or without tapping. Springs or other mechanical means including drilling with or without tapping followed by threaded screw insertion are also envisioned to deploy the end of the fastener once inserted into the ADR 40 so as to prevent withdrawal and support compression/stabilization of the ADR.

With the fastener in place, the relative movement of the ADR's endplates 42, 44 are restricted and the ADR is stabilized such that the subsequent spine fusion procedure and the rehabilitation/healing will not be frustrated by movement of the ADR. The inventor stresses that other fasteners or stabilizing techniques may be consistent with the present invention, and the invention should not be limited to only those described herein. Rather, it is envisioned that one of ordinary skill in the art given the Applicant's disclosure herein could devise equivalent fasteners that would work equally as well as those described herein and such alternative embodiments should be considered part of the present invention.

I claim:

1. A method for in situ stabilization of an artificial replacement disc located in an inter-vertebral disc space comprising the steps of:
using a blunt trocar and cannula to establish a pathway along an anterior sacrum;
advancing the blunt trocar and cannula to a position just below the disc space on the sacrum;
exchanging the blunt trocar for a sharp guide wire that is tapped into the sacrum;
dilating an entry of the sacrum up to approximately 10-12 mm with a working cannula docked to the sacrum;
passing a drill through the working cannula and dilated sacrum until it bears against an end plate of the ADR;
creating a through bore in the ADR using the drill; and
inserting a fastener into the bore, and compressing the ADR with the fastener.

2. The method of claim 1 wherein fluoroscopes oriented in orthogonal planes are used to position the drill at the ADR.

3. The method of claim 1 wherein irrigation of the drill during the bore creating step is performed.

4. The method of claim 1 wherein removal of debris from the drilling step is achieved using a vacuum at the disc space.

5. The method of claim 1 wherein the fastener includes a distal end including means for preventing the fastener from retracting through the ADR.

6. The method of claim 1 wherein the fastener includes a proximal end the cooperates with a distal end to compress the ADR therebetween.

7. The method of claim 1 wherein a distal end of the fastener umbrellas to prevent withdrawal of the fastener after insertion in the bore of the ADR.

8. The method of claim 1, where a postero lateral portal is created in addition to the pathway, for performing one of a group of steps including visual inspection of the inter-vertebral disc space with the ADR., irrigation of a region adjacent the drilling step, suction of debris resulting from said drilling step, and reduction, prior to stabilization, of any ADR dislocation or subluxation.
